(19) United States
(12) Patent Application Publication
(10) Pub. No.: US 2007/0213717 A1
(43) Pub. Date: Sep. 13, 2007

(54) Biological fusion in the vertebral column

(75) Inventors: Hai H. Trieu, Cordova, TN (US); Michael C. Sherman, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/354,312

(22) Filed: Feb. 14, 2006

(51) Int. Cl. A61F 2/30

(52) U.S. Cl. 606/61

(57) ABSTRACT

Methods are provided for treating a spinal condition. One method includes promoting fusion of an area of a vertebral column by introducing a biological treatment into the area to be fused. Another method includes causing damage in the area to be fused so as to induce a healing response. Other methods include applying a mechanical device to the vertebral column to provide stability to the area undergoing fusion.

### BACKGROUND

The present application relates generally to treatment of the vertebral column, for example, fusion between adjacent vertebrae.

Disease, degradation, and trauma of the spine can lead to various conditions that require treatment to maintain, stabilize, or reconstruct the vertebral column. For example, degeneration of the facet joints and/or the intervertebral discs due to aging and/or trauma can lead to pain, neurological deficit and/or loss of motions that require treatment to maintain, stabilize, reconstruct and/or regenerate the degenerated levels. One method for treatment includes providing stabilization by spinal fusion. Spinal fusion is a process in which an osseous bridge is formed between adjacent portions of the spinal column, such as adjacent vertebral bodies and/or endplates.

### SUMMARY

The present application relates generally to treatment of the vertebral column, for example, fusion between adjacent vertebrae. In some embodiments, fusion occurs between endplates of adjacent vertebrae or between vertebral bodies of adjacent vertebrae. In other embodiments, fusion occurs between facets of adjacent vertebrae.

In one embodiment, a method of treating a vertebral column includes promoting fusion in an area of a vertebral column. In one aspect, fusion across a joint between adjacent vertebrae is promoted by introducing a biological treatment into the joint. In some such embodiments, the joint is formed by adjacent facets or by adjacent vertebral bodies.

In other embodiments, fusion in a vertebral column is promoted or enhanced by causing damage to at least a portion of the area to be fused sufficient to induce a healing response. In some such embodiments, the facets and/or the endplates of adjacent vertebrae can be mechanically damaged sufficient to induce a healing response.

In yet another embodiment, a mechanical device can be applied to the vertebral column to provide stability during the fusion process. In some such embodiments, a mechanical device can be applied to any of an anterior region, an anterior column region, a posterior region or a spinous process region of the vertebral column.

In still other embodiments, a method for treating a motion segment of a vertebral column includes promoting fusion within an intact motion segment of a spinal column. In certain embodiments, the intact motion segment comprises an intact facet joint and/or intact adjacent vertebrae.

Additional embodiments are provided in the following description and the attached drawings.

### BRIEF DESCRIPTION OF THE DRAWINGS

FIG. 1 is a sagittal view of a motion segment of a vertebral column.

FIG. 2 is a superior view of a vertebral body depicted in FIG. 1.

FIGS. 3-4 illustrate methods for applying a biological treatment to a facet joint in a vertebral column.

FIGS. 5-6 illustrate methods for applying a biological treatment to a disc space in a vertebral column.

FIGS. 7A-7F illustrate methods for applying a biological treatment to a vertebral body and/or an endplate.

FIGS. 8A-8C illustrate alternative methods for applying a biological treatment to a vertebral body and/or an endplate.

FIG. 9 is a sagittal view of a motion segment of a vertebral column to which a biological treatment has been applied in combination with a mechanical device.

### DETAILED DESCRIPTION

The present disclosure relates generally to treatment of the vertebral column, for example, fusion between adjacent vertebrae. As discussed herein, fusion is a process in which an osseous bridge is formed between adjacent bony portions of the spinal column, such as adjacent vertebral bodies, endplates, and facets.

Adjacent vertebrae comprise a motion segment of the spinal column. Each vertebra comprises a facet, a vertebral body with superior and inferior endplates, and in intervertebral disc. Thus, fusion between adjacent vertebrae includes any of fusion within a motion segment, fusion across a facet joint, fusion between adjacent vertebral bodies, or fusion between adjacent endplates. As described herein, fusion between adjacent vertebrae is promoted using a mechanical approach, a biological approach, or various mechanical approaches in combination with a biological approach.

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

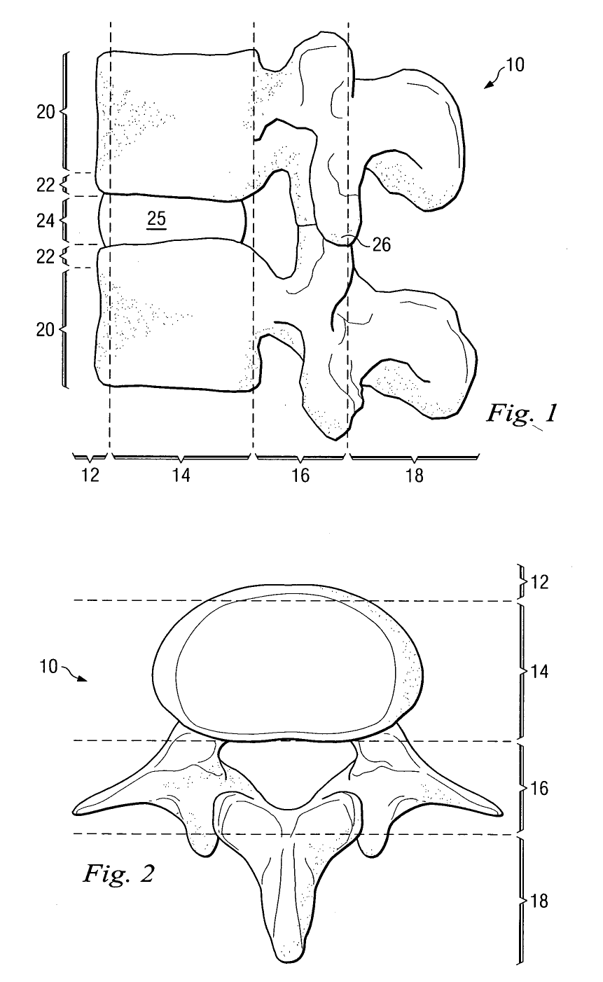

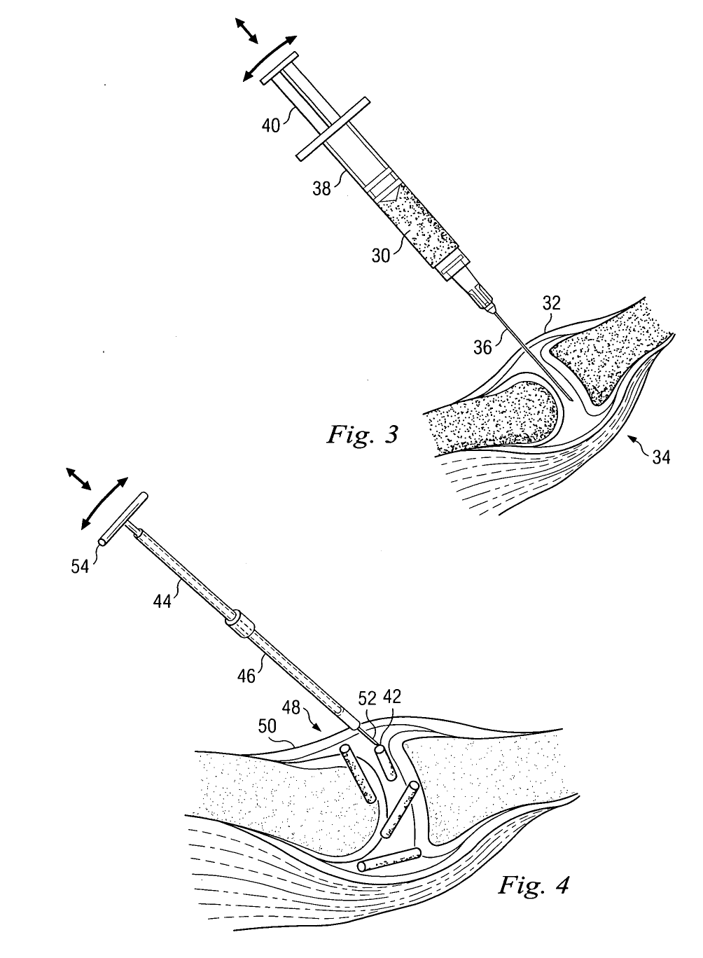

Referring now to FIGS. 1 and 2, the reference numeral 10 refers to a motion segment of a vertebral column. Motion segment 10 comprises an intervertebral disc 25 and a facet joint 26. Motion segment 10 may be considered as having several regions extending from anterior to posterior. These regions include an anterior region 12, an anterior column region 14, a posterior region 16, and a spinous process region 18. The anterior column region 14 may be further considered to have several regions extending longitudinally along the column. These regions include a vertebral body region 20, an endplate region 22, and a disc space region 24. Disc space region 24 includes the nucleus and annulus forming intervertebral disc 25.

Any of the regions illustrated in FIGS. 1 and 2 may be treated by fusion as described herein. In certain embodiments, fusion of a facet joint is performed using a mechanical approach that includes gaining access to the facet joint sufficient to allow for contact between a tool and the inferior and/or superior facet. In similar embodiments, fusion across a disc space is performed by gaining access to the disc space sufficient to allow for contact between a tool and the inferior and/or superior endplates, or the adjacent vertebral bodies. In either embodiment, the tool will be used to cause damage to the facet, endplate or vertebral body so as to induce a healing response, and so will be provided with a sharp tip, or with serrations, or with a blade, or with other means for cutting, scraping, or otherwise damaging the facet, endplate or vertebral body to a degree sufficient to induce a healing response. In certain embodiments, the tool may be a curette or a chisel, and will be manipulated so as to cause bleeding of the facet, endplate or vertebral body, thereby inducing a healing response.

In other embodiments, fusion of a facet joint or other area of the vertebral column is achieved with a biological approach that includes application of a biological treatment to the facet joint or desired area, wherein the biological treatment includes materials that promote fusion. The biological treatment can be applied to the desired area using various access methods. For example, the biological treatment can be applied to the desired area using either an open procedure or a minimally invasive procedure. In other examples, the biological treatment can be injected into the desired area either percutaneously or through a needle.

In still other embodiments, fusion of a facet joint or other area of the vertebral column is achieved by a mechanical approach in combination with a biological approach. For example, a tool used to damage a facet as described above may be combined with application of biological treatment to the facet joint. As another example, a mechanical device may be applied to the vertebral column while a biological treatment is applied to a facet joint or other area of the vertebral column. The mechanical device provides for stability during the fusion process, which is promoted by the biological treatment.

As used herein, a “biological treatment” will include materials that promote fusion of vertebral bone, for example, the facets or the vertebral endplates. Such a biological treatment includes but is not limited to a “biologically active component”, with or without a “biological additive”.

A “biologically active component” includes but is not limited to anti-cytokines, anti-interleukin-1 components (anti-IL-1); anti-TNF alpha; “growth factors”; LIM mineralization proteins; “stem cell material”, osteoblasts, and cells containing a viral vector for osteoinductivity. The acronym “LIM” is derived from the three genes in which the LIM domain was first described. The LIM domain is a cysteine-rich motif defined by 50-60 amino acids with the consensus sequence CX₂CX₁₆₋₂₃HX₂CX₂CX₂CX₁₆₋₂₁CX₂(C/H/D), which contains two closely associated zinc-binding modules. LIM mineralization proteins include but are not limited to those described in U.S. Patent Application Publication No. 2003/0180266 Al, the disclosure of which is incorporated herein by reference. “Growth factors” include but are not limited to bone morphogenetic protein (BMP)-2, BMP-3, BMP-4, BMP-6, BMP-7, BMP-9; platelet derived growth factor (PDGF); insulin-like growth factor (ILGF); human endothelial cell growth factor (ECGF); nerve growth factor (NGF); and vascular endothelial growth factor (VEGF). “Anti-IL-1” components include but are not limited to those described in U.S. Patent Application Publication Nos. 2003/0220283 and 2005/0260159, the entire disclosures of which are incorporated herein by reference. “Stem cell material” includes but is not limited to dedifferentiated stem cells, undifferentiated stem cells, and mesenchymal stem cells. “Stem cell material” also includes but is not limited to stem cells extracted from marrow, which may include lipo-derived stem cell material, and adipose-derived stem cell material, such as described in U.S. Publication Nos. 2004/0193274 and 2005/0118228, each of which is incorporated herein by reference. “Stem cell material” also includes but is not limited to stem cells derived from adipose tissue as described in U.S. Patent Application Publication Nos. 2003/0161816, 2004/0097867 and 2004/0106196, each of which is incorporated herein by reference.

A “biologically active component” also includes but is not limited to an activated tissue graft, such as described in U.S. Patent Application Publication No. 2005/0136042, the entire disclosure of which is incorporated herein by reference; an engineered cell comprising a nucleic acid for encoding a protein or variant thereof, such as a BMP, a LIM mineralization protein, or an SMAD protein as described in U.S. Patent Application Publication Nos. 2003/0219423 and 2003/0228292, the entire disclosures of which are incorporated herein by reference; and a recombinant human bone morphogenetic protein, such as described in U.S. Patent Application Publication No. 2004/0024081, the entire disclosure of which is incorporated herein by reference.

As used herein, a “biological additive” includes but is not limited to “biomaterial carriers”, “therapeutic agents”, “liquids” and “lubricants.”

“Biomaterial carriers” include but are not limited to collagen, gelatin, hyaluronic acid, fibrin, albumin, keratin, silk, elastin, calcium phosphate, calcium sulfate, glycosaminoglycans (GAGs), polyethylene glycol (PEG), polyethylene oxide (PEO), polyvinyl alcohol (PVA) hydrogel, polyvinyl pyrrolidone (PVP), co-polymers of PVA and PVP, other polysaccharides, platelet gel, peptides, carboxymethyl cellulose, and other modified starches and celluloses. Collagen includes but is not limited to collagen-based material, which may be autogenic, allogenic, xenogenic or of human-recombinant origin, such as the collagen-based material described in U.S. Patent Application Publication Nos. 2004/0054414 and 2004/0228901, the entire disclosures of which are incorporated herein by reference.

“Therapeutic agents” include but are not limited to analgesics, antibiotics, anti-inflammatories, steroids, antiviricides, vitamins, amino acids and peptides. Analgesics include but are not limited to hydrophilic opoids, such as codeine, prodrugs, morphine, hydromorphone, propoxyphene, hydrocodone, oxycodone, meperidine and methadone, and lipophilic opoids, such as fentanyl. Antibiotics include but are not limited to erythromycin, bacitracin, neomycin, penicillin, polymyxin B, tetracyclines, viomycin, chloromycetin and streptomycins, cefazolin, ampicillin, azactam, tobramycin, clindamycin and gentamycin.

“Liquids” include but are not limited to water, saline and radio-contrast media. Radio-contrast media includes but is not limited to barium sulfate, or a radio contrast dye, such as sodium diatrizoate (HYPAQUE™).

“Lubricants” include but are not limited to hyaluronic acid, a salt of hyaluronic acid, sodium hyaluronate, glucosaminoglycan, dermatan sulfate, heparin sulfate, chondroitin sulfate, keratin sulfate, synovial fluid, a component of synovial fluid, vitronectin and rooster comb hyaluronate.

A biological treatment may be introduced to an area of a vertebral column by any method and in any form appropriate for such introduction. For example, the biological treatment can be injected, deposited, or applied, as a solution, a suspension, emulsion, paste, a particulate material, a fibrous material, a plug, a solid, porous, woven or non-woven material, or in a dehydrated or rehydrated state. Suitable forms for a biological treatment and suitable methods for injecting a biological treatment include those described in U.S. Patent Application Publication Nos. 2005/0267577, 2005/0031666, 2004/0054414, and 2004/0228901, each of which is incorporated herein by reference.

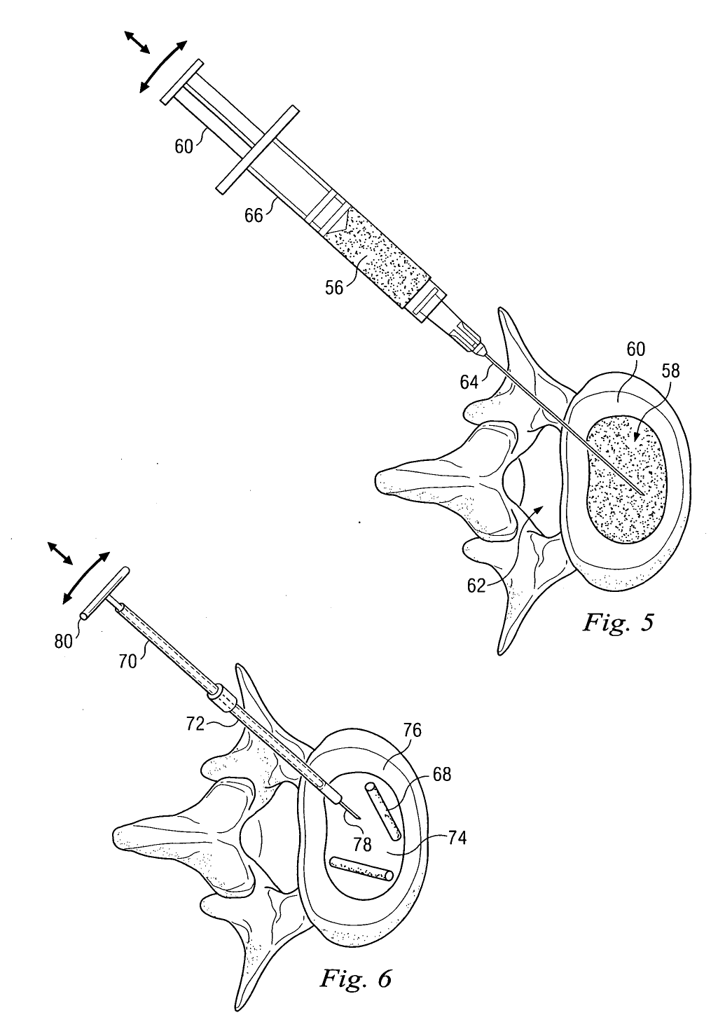

For example, referring now to FIG. 3, a biological treatment 30 may be injected into the joint capsule 32 of a facet joint 34 through a hypodermic needle 36 attached to a syringe 38. In some embodiments, a biological treatment is applied to an intact facet joint. In one such embodiment, a syringe is inserted through the vertebral annulus, which is the connective tissue between adjacent vertebrae, through the joint capsule, and into the space between the adjacent facets, and/or into contact with the inferior and superior surfaces of the adjacent facets. As illustrated in FIG. 3, syringe 38 is so inserted into the joint capsule 32, and the syringe plunger 40 is depressed, thereby releasing the biological treatment into the joint capsule of the facet joint. As illustrated by the arrows in FIG. 3, the needle/syringe assembly may be moved around within the joint capsule, sweeping from side to side and back and forth, to ensure uniform distribution of the biological treatment within the facet joint. It is preferred, however, that the tip of the needle be maintained near the center of the joint capsule to ensure deposition of the material within the desired area, and to minimize potential leakage.

Referring now to FIG. 4, another method for injecting a biological treatment into a facet joint is illustrated. According to the embodiment illustrated in FIG. 4, a biological treatment 42 is provided in the form of microspheres, powders, particulates, pellets, granules, a plug, a solid, porous, woven or non-woven material. Biological treatment 42 may be compressed into a size suitable for delivery through a cannula 44 by pressure and/or heat and/or insertion through a small diameter tube. The delivery cannula 44 is attached to a dilator 46. The biological treatment 42 is inserted into a facet joint 48 by penetrating the capsule 50 of the facet joint with a guide needle 52. Dilator 46, preferably with delivery cannula 44 already attached, is inserted over guide needle 52. A plunger 54 may be used to push the biological treatment from the cannula into the facet joint. The form of the biological treatment may expand upon exiting the dilator, and may further expand as it hydrates or rehydrates in the facet joint. Such forms of biological treatments can also be applied through the vertebral annulus to an intact facet joint.

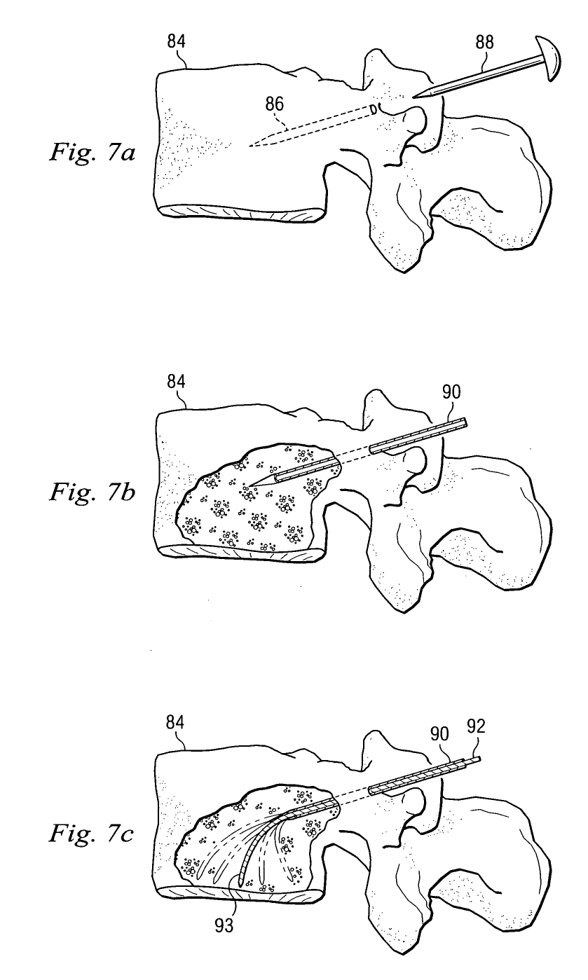

Similar methods as described with respect to FIGS. 3 and 4 can be used to inject a biological treatment into a disc space, vertebral bodies and endplates. Referring now to FIG. 5, a method for injecting a biological treatment into a disc space is illustrated. In some embodiments where fusion of vertebral bodies, endplates, or across a disc space is desired, a biological treatment can be injected into an intact disc, vertebral body or endplate through the vertebral annulus, through the annulus of the disc, and into the disc space. According to the embodiment illustrated in FIG. 5, a biological treatment 56 may be injected into the nucleus pulposus 58 contained within a disc annulus 60 in an intervertebral disc space 62. Biological treatment 56 is injected through a hypodermic needle 64 attached to a syringe 66. The syringe 66 is inserted into the nucleus pulposus, and the syringe plunger 68 is depressed, thereby releasing the biological treatment into the disc space 62. As illustrated by the arrows in FIG. 5, the needle/syringe assembly may be moved around, sweeping from side to side and back and forth, to ensure uniform distribution of the biological treatment within the disc space. It is preferred, however, that the tip of the needle be maintained near the center of the disc space to ensure deposition of the material within the nucleus of the disc, and to minimize potential leakage.

Referring now to FIG. 6, another method for injecting a biological treatment into a disc space is illustrated. According to the embodiment illustrated in FIG. 6, a biological treatment 68 is provided in the form of granules, a plug, a solid, porous, woven or non-woven material. Biological treatment 68 may be compressed into a size suitable for delivery through a cannula 70 by pressure and/or heat and/or insertion through a small diameter tube. The delivery cannula 70 is attached to a dilator 72. The biological treatment 68 is inserted into the nucleus pulposus 74 by penetrating the annulus 76 of the disc with a guide needle 78. Dilator 72, preferably with delivery cannula 70 already attached, is inserted over guide needle 78. A plunger 80 may be used to push the biological treatment from the cannula into the nucleus pulposus. The form of the biological treatment may expand upon exiting the dilator, and may further expand as it hydrates or rehydrates. Such forms of biological treatments can also be inserted through the vertebral annulus and into an intact disc, vertebral body or endplate.

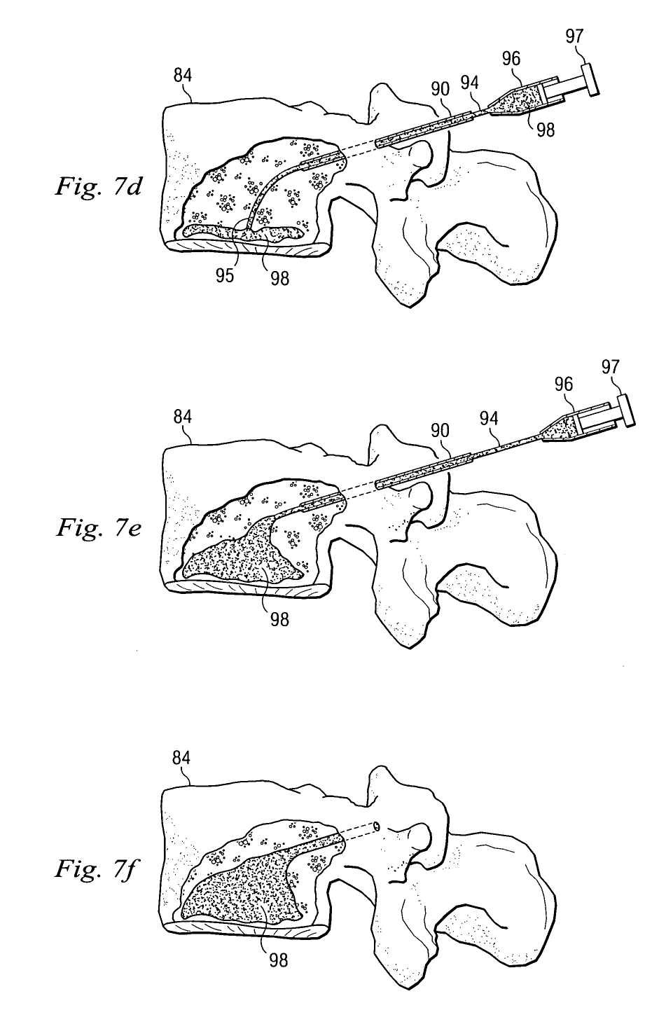

Referring now to FIGS. 7A-7F, a method of injecting a biological treatment into a vertebral body and/or an endplate is illustrated. A channel 86 can be created in vertebral body 84 through the pedicle using a suitable bone-penetrating implement such as a trocar needle 88. (FIG. 7A). A sheath 90 can be inserted into channel 86 through which various procedures can be implemented. (FIG. 7B). FIG. 7C shows a subsequent step in which a flexible or otherwise steerable device 92, such as a needle or drill, is positioned through sheath 90 to access regions nearing the endplate of vertebral body 84. Although FIG. 7C illustrates positioning sheath 90 to access regions near the endplate of vertebral body 84, sheath 90 could also be positioned so as to access regions more central to the vertebral body itself, as opposed to the endplate.

Referring still to FIG. 7C, several directional passes of the steerable device 92 may be used in order to create access to a broader volume of bone. The tip 93 of steerable device 92 can be designed so as to be steerable, for instance by rotation of steerable device 92. As illustrated in FIG. 7D, after accessing near the endplate, (or to the vertebral body itself in other embodiments), the steerable device 92 can be withdrawn, and a delivery device 94 can be inserted through sheath 90. Delivery device 94 can have delivery tip 95, which is curved or otherwise steerable. Delivery device 94 can also include a reservoir 96 and a plunger 97, allowing for the delivery of a biological treatment 98 out of delivery tip 93. FIG. 7E shows an intermediate stage of the delivery process in which additional amounts of the biological treatment 98 are delivered as the sheath 90 and the delivery device 94 are withdrawn from the access channel 86. In this manner, the access channel 86 can be backfilled with the biological treatment 98 as the implements are withdrawn. Finally, shown in FIG. 7F is the biological treatment 98 occupying a volume overlying an endplate of the vertebral body 84, and also backfilled into the access channel 86.

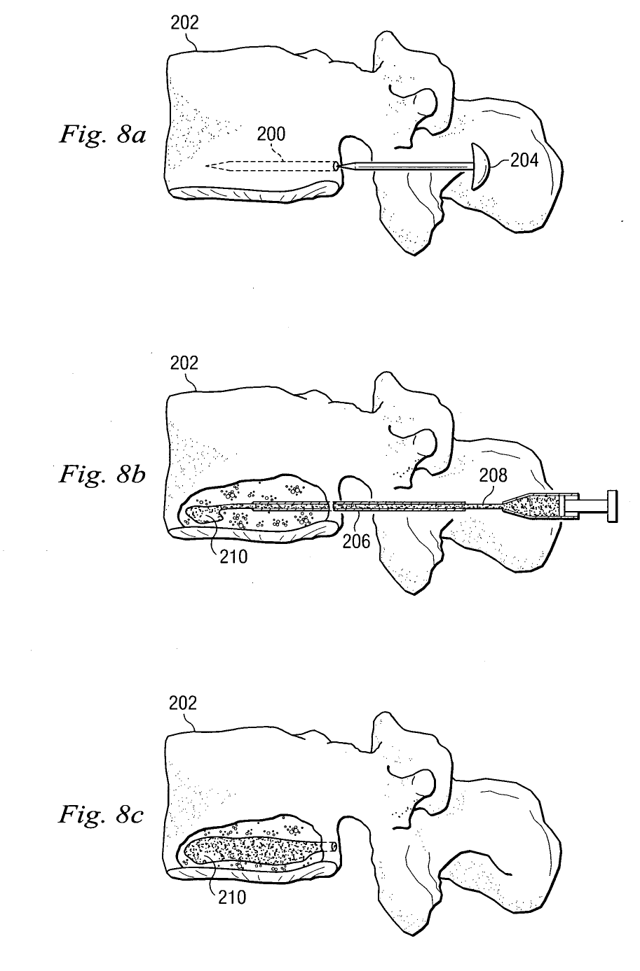

Referring now to FIGS. 8A-8C, another method of injecting a biological treatment into a vertebral body and/or an endplate is illustrated. An access channel 200 is created in vertebral body 202 just above the endplate using a bone-penetrating implement 204, for example, a needle. (FIG. 8A). After this access, a sheath 206 is provided into channel 200. (FIG. 8B). A delivery device 208 is then inserted through the lumen of sheath 206 and is used to deliver a biological treatment 210 into the vertebral body in a volume overlying the endplate. If desired or needed, a steerable needle or drill can be used to create access to a broader volume of bone, generally as described in conjunction with FIGS. 7A through 7F above. As well, a backfilling procedure can be used to fill the access channel 200 as the delivery device 208 and sheath 206 are removed. As shown in FIG. 8C, ultimately, a volume of the biological treatment 210 is delivered into the vertebral body overlying the endplate.

In other embodiments, a biological treatment may be introduced into an area of a vertebral column, such as a motion segment, through a needle/trocar assembly, as described in the above-referenced U.S. Patent Application Publication Nos. 2005/0031666. In still other embodiments, a biological treatment may be introduced into an area of a vertebral column by extrusion through a dilated annular opening, infusion through a catheter, insertion through an opening created by trauma or surgical incision, or by other means of invasive or minimally invasive deposition of materials into the area receiving the biological treatment.

Thus, methods are provided herein to achieve fusion of one or more of a disc space, vertebral bodies, end plates, and facet joints, using a mechanical approach, a biological approach, or various mechanical approaches in combination with a biological approach.

A mechanical approach includes gaining access to the area of the vertebral column to be fused sufficient to allow for damage to be caused in the area. For example, if a facet joint is to be fused, then sufficient access to the vertebral column would allow for contact between a tool and the inferior and/or superior facet. Such a mechanical approach further includes damaging the area to be fused with the tool so as to induce a healing response. A biological approach includes applying a biological treatment to the area to be fused, wherein the biological treatment includes materials that promote fusion. In embodiments where a facet joint is to be fused, the biological treatment may be applied anywhere in the facet joint, for example, the surfaces of the inferior and/or superior facet, and/or the joint space between the inferior and superior facets. The biological treatment may be injected into the facet joint or other area of the vertebral column by a suitable method, such as the methods illustrated in FIGS. 3-8.

According to one embodiment of a combined approach, a biological treatment sufficient to promote fusion is applied to the facet joint, and a mechanical device is applied to at least one of the anterior region, the anterior column region, the posterior region, or the spinous process region of the spine. According to another embodiment, a biological treatment sufficient to promote fusion is applied to the vertebral body and/or the endplates, and a mechanical device is applied to at least one of the anterior region, the posterior region, or the spinous process region of the spine.

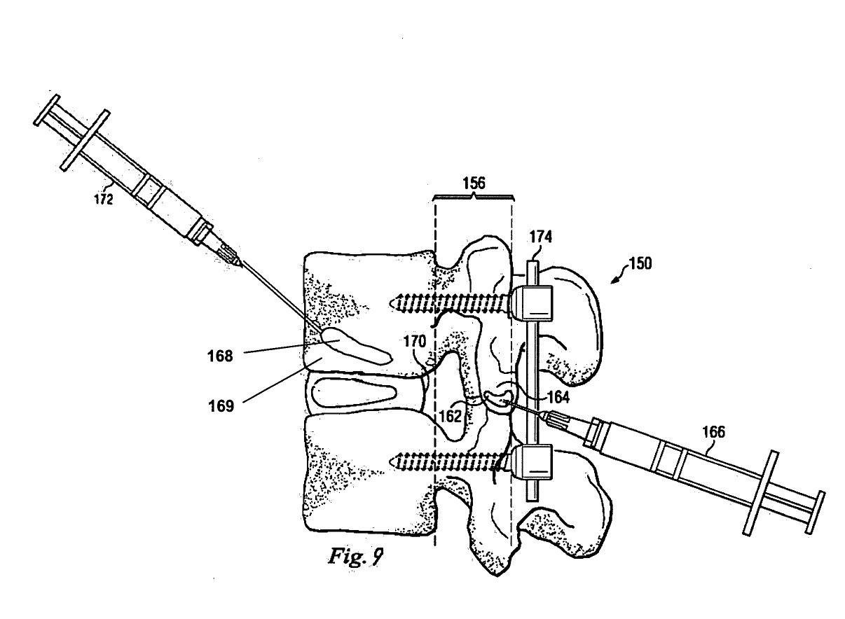

Referring now to FIG. 9, a biological treatment 162 has been applied to facet joint 164 by injection with an appropriately sized hypodermic needle 166. Selection of an appropriately sized hypodermic needle for injection into the facet joints of a spine is within the purview of one of ordinary skill in the art. Suitable methods for injecting the biological treatment 162 into the facet joint 164 include those described above with respect to FIGS. 3 and 4. Other methods as described herein and as are known to those of ordinary skill in the art may also be used.

In the embodiment illustrated in FIG. 9, a biological treatment 168 has also been applied to vertebral body 169, which could include treatment of either or both of the vertebral body and the endplate, with an appropriately sized hypodermic needle 172. Selection of an appropriately sized hypodermic needle for injection into the disc space of a spine is within the purview of one of ordinary skill in the art. Suitable methods for injecting the biological treatment 168 into the vertebral body 169 are described above with respect to FIGS. 7 and 8. Other methods as described herein and as are known to those of ordinary skill in the art may also be used.

Although two biological treatments 162 and 168 are illustrated, the present disclosure contemplates and includes application of just one biological treatment, or of two or more biological treatments. For example, a biological treatment could be applied to only the facet joint 164, or alternatively, to only the vertebral body 169. Moreover, biological treatments can be applied in one or more of the anterior longitudinal ligament, and the disc space 170, which includes the disc annulus and the nucleus pulposus.

Biological treatments 162 and 168 can be any of the materials described herein. In certain embodiments, a biological treatment includes at least one of BMP-2, BMP-7, and LIM protein. A suitable BMP-2 includes but is not limited to INFUSE brand products commercially available from Medtronic. In some embodiments, a biological treatment includes at least one of collagen, hydroxyapatite, calcium phosphate, demineralized bone matrix (DBM), or combinations thereof.

According to the embodiment illustrated in FIG. 9, treatment of facet joint 164 and vertebral body 169 with biological treatments 162 and 168 is combined with a posterior device applied to the posterior region 156 of the vertebral motion segment 150. The posterior device is represented in FIG. 9 by posterior device 174, however the appearance of posterior device 174 is illustrative only, and it is understood that a wide variety of posterior devices could be used with the present embodiments. Moreover, mechanical devices can be applied to regions of the vertebral column other than the posterior region as illustrated in FIG. 9. For example, mechanical devices can be applied to the anterior region or the spinous process region of the vertebral column to provide stability to the area being fused.

According to some embodiments, a posterior device 174 may extend along the posterior or posterolateral side of the vertebral column and may span one or more vertebral motion segments.

In other embodiments, a posterior device 174 may be a rigid fixation system such as a hook, rod, or screw system, which are offered by or developed by Medtronic, Inc. of Minneapolis, Minn. under brands such as CD HORIZON, CD HORIZON SEXTANT, CD HORIZON M8, CD HORIZON LEGACY, CD HORIZON ANTARES, COLORADO 2, EQUATION, VERTEX, TSRH, TSRH-3D, KOBRA, and VERTELINK SST. Other suitable posterior devices include certain devices offered by Trans1, Inc. (formerly “Axiamed”).

In yet other embodiments, a posterior device 174 may be a semi-rigid or flexible system offered by or developed by Medtronic, Inc. under brand names such as FLEXTANT or AGILE, or offered by or developed by Zimmer, Inc. of Warsaw, Ind. such as the Dynesys® Dynamic Stabilization System. These types of flexible systems may be disclosed, for example, in U.S. Pat. Pub. Nos. 2005/0171540 and 2005/0131405. These particular systems may attach to the posterior features of adjacent vertebrae using bone screws.

According to still other embodiments, a posterior device 174 may be a dampener system, such as those described in U.S. Pat. Nos. 5,375,823; 5,540,688; 5,480,401 or U.S. Pat. App. Pub. Nos. 2003/0055427 and 2004/0116927, each of which is incorporated by reference herein.

In still another embodiment, posterior device 174 may include annulus repair or replacement devices for the posterior portion of the annulus. Additionally, posterior device 174 may also be a rod and screw system that uses flexible PEEK rods.

In still other embodiments, posterior device 174 may be made of flexible materials, such as woven or braided textile based devices that connect with two or more vertebrae. These flexible materials may be formed of natural graft material or synthetic alternatives. Posterior device 174 may also be formed of inelastic material, such as braided tethers or woven fabric of polyester or polyethylene, or of elastic material, such as rubber banding or plates, sheets, rods, or tubing made of silicone or polyurethane.

Posterior device 174 may be formed from biocompatible materials such as metals, polymers, ceramics, and tissue, and combinations thereof. For example, posterior device 174 may be formed from rigid materials such as a titanium, stainless steel, titanium alloy, nickel titanium, or tantalum. Alternatively, posterior device 174 may be formed of less rigid or more flexible materials such as polyaryletherketone (PAEK)-based materials, which includes polyetheretherketone (PEEK), polyetherketoneketone (PEKK), PEEK-carbon composite, etc., polyetherimide, polyimide, polysulfone, polyethylene, polyester, polylactide, copolymers of poly L-lactide and poly D-lactide, polyorthoester, tyronsine polycarbonate, polypolyurethane, silicone, etc. In some embodiments, the posterior device may be bioresorbable or partially resorbable.

Posterior device 174 may be connected to two or more vertebral bodies or vertebral endplates through the use of any connection mechanism such as bone screws, staples, sutures, or adhesives. The posterior device may be loaded in compression or tension depending upon the patient's indication or the performance of other implanted systems or treatments. For example, a flexible posterior device attached to adjacent vertebrae with bone screws may be installed in compression to stabilize the vertebral column, including the facet joint 164 where a biological treatment was applied.

Any of the foregoing posterior devices may be combined with any biological treatment applied to the facet joint or other areas of the vertebral column.

Any of the regions illustrated in FIGS. 1 and 2 may be treated by fusion as described herein. In certain embodiments, fusion of a facet joint is performed using a mechanical approach that includes gaining access to the facet joint sufficient to allow for contact between a tool and the inferior and/or superior facet. Such access can be gained by percutaneous insertion of the tool into the facet joint, or by an open procedure in which at least a portion of the patient's vertebral column is surgically exposed, or by combinations of percutaneous and open procedures. Suitable percutaneous procedures, open procedures, and combinations thereof are known to those of ordinary skill.

According to one embodiment of a mechanical approach, a tool is used to cause damage to the facet. Such a tool will be provided with a sharp tip, or with serrations, or with a blade, or with other means for cutting, scraping, or otherwise damaging the facet to a degree sufficient to induce a healing response. In certain embodiments, the tool may be a curette or a chisel, and will be manipulated so as to cause bleeding of the facet, thereby inducing a healing response. Other areas of the vertebral column, for example, the endplates, may also be fused with a mechanical approach as described herein.

In other embodiments, fusion of a facet joint is achieved by applying a biological treatment to the facet joint, wherein the biological treatment includes materials that promote fusion. Other areas of the vertebral column, for example, the endplates, may also be fused with a biological approach as described herein.

In still other embodiments, fusion of a facet joint is achieved by damaging the facets so as to induce a healing response, and applying a biological treatment to the facets joint so as to enhance the healing response and the resulting fusion of the facet joint. Other areas of the vertebral column, for example, the endplates, may also be fused with a combined biological and mechanical approach as described herein.

In still further embodiments, a mechanical device may be applied to the posterior region of the vertebral column while a biological treatment is applied to the area to be fused. According to one such embodiment, at least a portion of the patient's spine is surgically accessed, and the mechanical device is implanted at a desired location. A biological treatment is then applied to the area to be fused. These steps may be reversed such that the biological treatment is applied first, and the mechanical device is applied later.

In certain embodiments, the mechanical device is implanted into an area of the spine that is intact, for example, a motion segment where the anatomy has not been surgically disrupted. In another aspect, the anatomy of the area of the spine in which the mechanical device is being implanted has been surgically disrupted, for example, a resection of the spinous process, or even a discectomy, has been performed.

In other embodiments, regardless of whether the spinal anatomy is intact or has been disrupted, the mechanical device is implanted into the spine in a position so as to provide stability with respect to the area that is receiving a biological treatment. In one aspect, the facet joints and/or the adjacent vertebral bodies defining the disc space are mechanically moved by placement of the mechanical device to align the facet joint and/or increase the distance between the adjacent vertebral bodies. After application of the mechanical device, a biological treatment is applied to the facet joint or other area of the vertebral column selected for biological treatment. In another aspect, the above-described steps may be reversed such that the biological treatment of the facet joint or other area occurs first, and the mechanical device is applied later.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

For example, each of the following patent applications are incorporated herein by reference, as each describes spinal devices that can be applied to the anterior, anterior column, posterior, or spinous process regions of the vertebral column, and that can be used to unload an area treated with a biological treatment as described herein.

| | Attorney | | |
|---|---|---|---|
| Title | Docket No. | Filing Date | Inventor(s) |
| Materials, Devices, and Methods for | P22656.00 | Jan. 13, | Hai H. Trieu |
| Treating Multiple Spinal Regions | 31132.378 | 2006 | |
| Including The Interbody Region | | | |
| Materials, Devices, and Methods for | P22578.00 | Jan. 13, | Hai H. Trieu |
| Treating Multiple Spinal Regions | 31132.376 | 2006 | |
| Including The Posterior and Spinous | | | |
| Process Regions | | | |
| Materials, Devices, and Methods for | P22615.00 | Jan. 13, | Hai H. Trieu |
| Treating Multiple Spinal Regions | 31132.377 | 2006 | |
| Including The Anterior Region | | | |
| Materials, Devices, and Methods for | P22681.00 | Jan. 13, | Hai H. Trieu |
| Treating Multiple Spinal Regions | 31132.379 | 2006 | |
| Including Vertebral Body and Endplate | | | |
| Regions | | | |
| Use Of A Posterior Dynamic | P22397.00 | Jan. 13, | Aure Bruneau et al. |
| Stabilization System With An | 31132.420 | 2006 | |
| Interdiscal Device | | | |

In addition, each of the following applications describes suitable biological treatments that can be applied to an area of the vertebral column, and spinal devices that can be applied to the anterior, anterior column, posterior, or spinous process regions of the vertebral column to unload the treated area. Each of the following applications was filed concurrently with the present application, assigned to the same assignee, and each is hereby incorporated by reference.

| | Attorney | | |
|---|---|---|---|
| | Docket | | |
| Title | No. | Filing Date | Inventor(s) |
| Treatment of the Vertebral | P23559.00 | concurrent with | Hai H. Trieu |
| Column | 31132.477 | the present | |
| | | application | |
| Treatment of the Vertebral | P23556.00 | concurrent with | Hai H. Trieu |
| Column | 31132.474 | the present | |
| | | application | |
| Treatment of the Vertebral | P23558.00 | concurrent with | Hai H. Trieu |
| Column | 31132.476 | the present | |
| | | application | |
| Treatment of the Vertebral | P23557.00 | concurrent with | Hai H. Trieu |
| Column | 31132.475 | the present | |
| | | application | |
| Treatment of the Vertebral | P23598.00 | concurrent with | Hai H. Trieu |
| Column | 31132.479 | the present | |
| | | application | |

It is understood that all spatial references, such as “horizontal,” “vertical,” “top,” “inner,” “outer,” “bottom,” “left,” “right,” “anterior,” “posterior,” “superior,+ “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method of causing fusion across a facet joint of a vertebral column comprising:
injecting a biological treatment into the facet joint to promote fusion across the facet joint.

2. The method of claim 1 further comprising:
causing damage to at least a portion of the facet joint sufficient to induce a healing response in the facet joint.

3. The method of claim 1 further comprising applying a mechanical device to the vertebral column.

4. The method of claim 3 further comprising:
causing damage to at least a portion of the facet joint sufficient to induce a healing response in the facet joint.

5. The method of claim 3 wherein the mechanical device is applied to a posterior region of the vertebral column.

6. The method of claim 3 wherein the mechanical device is formed from a biocompatible material selected from metals, polymers, ceramics, and tissue, and combinations thereof.

7. The method of claim 6 wherein the mechanical device is formed from a metal selected from titanium, titanium alloys, nickel titanium, tantalum, stainless steel, and combinations thereof.

8. The method of claim 6 wherein the mechanical device is formed from a material selected from polyaryletherketone (PAEK), polyetheretherketone (PEEK), polyetherketoneketone (PEKK), PEEK-carbon composite, polyetherimide, polyimide, polysulfone, polyethylene, polyester, polylactide, copolymers of poly L-lactide and poly D-lactide, polyorthoester, tyrosine polycarbonate, polyurethane, silicone, polyolefin rubber, and combinations thereof.

9. The method of claim 3 wherein the mechanical device is bioresorbable or partially resorbable.

10. The method of claim 1 wherein the biological treatment comprises a biologically active component.

11. The method of claim 10 wherein the biological treatment further comprises a biological additive.

12. The method of claim 11 wherein the biological additive comprises at least one of a biomaterial carrier, a therapeutic agent, a liquid and a lubricant.

13. The method of claim 11 wherein the biological additive is selected from autogenic collagen, allogenic collagen, xenogenic collagen, human recombinant collagen, gelatin, hyaluronic acid, fibrin, albumin, keratin, silk, elastin, calcium phosphate, calcium sulfate, glycosaminoglycans (GAGs), polyethylene glycol (PEG), polyethylene oxide (PEO), polyvinyl alcohol (PVA) hydrogel, polyvinyl pyrrolidone (PVP), co-polymers of PVA and PVP, polysaccharides, platelet gel, peptides, carboxymethyl cellulose, modified starches and celluloses.

14. The method of claim 11 wherein the biological additive is selected from analgesics, antibiotics, anti-inflammatories, steroids, antiviricides, vitamins, amino acids and peptides.

15. The method of claim 14 wherein the biological additive comprises at least one of:
an analgesic selected from codeine, prodrugs, morphine, hydromorphone, propoxyphene, hydrocodone, oxycodone, meperidine, methadone, and fentanyl; and
an antibiotic selected from erythromycin, bacitracin, neomycin, penicillin, polymyxin B, tetracyclines, viomycin, chloromycetin, streptomycins, cefazolin, ampicillin, azactam, tobramycin, clindamycin and gentamycin.

16. The method of claim 11 wherein the biological additive is selected from water, saline, radio-contrast media, hyaluronic acid, a salt of hyaluronic acid, sodium hyaluronate, glucosaminoglycan, dermatan sulfate, heparin sulfate, chondroitin sulfate, keratin sulfate, synovial fluid, a component of synovial fluid, vitronectin and rooster comb hyaluronate.

17. The method of claim 1 wherein the biological treatment comprises a biologically active component selected from anti-cytokines; anti-interleukin-1 components (anti-IL-1); anti-TNF alpha; growth factors; LIM mineralization proteins; stem cell material, osteoblasts and cells containing a viral vector for osteoinductivity.

18. The method of claim 1 wherein the biological treatment comprises a biologically active component selected from bone morphogenetic proteins, platelet derived growth factor (PDGF), insulin-like growth factor (ILGF), human endothelial cell growth factor (ECGF), nerve growth factor (NGF), and vascular endothelial growth factor (VEGF).

19. The method of claim 18 wherein the biologically active component comprises at least one bone morphogenetic protein selected from BMP-2, BMP-3, BMP-4, BMP-6, BMP-7, and BMP-9.

20. The method of claim 1 wherein the biological treatment comprises stem cell material selected from dedifferentiated stem cells, undifferentiated stem cells, mesenchymal stem cells, marrow-extracted stem cell material and adipose-derived stem cell material.

21. The method of claim 1 wherein the biological treatment comprises a biologically active component selected from biologic tissues, activated tissue grafts, engineered cells comprising a nucleic acid for encoding a protein or variant thereof, and a recombinant human bone morphogenetic protein.

22. A method of promoting fusion in a joint between adjacent vertebrae comprising:
accessing a joint between adjacent vertebrae in a vertebral column; and
introducing a biological treatment into the joint between the adjacent vertebrae to promote fusion across the joint.

23. The method of claim 22 wherein introducing the biological treatment into the joint comprises injecting the biological treatment into the joint.

24. The method of claim 23 wherein injecting the biological treatment into the joint comprises at least one of a percutaneous injection and injection through a needle.

25. The method of claim 23 wherein fusion is promoted across at least one of a facet joint and adjacent vertebral bodies.

26. The method of claim 22 further comprising applying a mechanical device to the vertebral column.

27. The method of claim 26 wherein the mechanical device is applied to at least one of an anterior region, a posterior region, and a spinous process region of the vertebral column.

28. The method of claim 22 wherein the biological treatment comprises a biologically active component.

29. The method of claim 28 wherein the biological treatment further comprises a biological additive.

30. The method of claim 29 wherein the biological additive comprises at least one of a biomaterial carrier, a therapeutic agent, a liquid and a lubricant.

31. The method of claim 30 wherein the biological additive is selected from autogenic collagen, allogenic collagen, xenogenic collagen, human recombinant collagen, gelatin, hyaluronic acid, fibrin, albumin, keratin, silk, elastin, calcium phosphate, calcium sulfate, glycosaminoglycans (GAGs), polyethylene glycol (PEG), polyethylene oxide (PEO), polyvinyl alcohol (PVA) hydrogel, polyvinyl pyrrolidone (PVP), co-polymers of PVA and PVP, polysaccharides, platelet gel, peptides, carboxymethyl cellulose, modified starches and celluloses.

32. The method of claim 30 wherein the biological additive is selected from analgesics, antibiotics, anti-inflammatories, steroids, antiviricides, vitamins, amino acids and peptides.

33. The method of claim 32 wherein the biological additive comprises at least one of:
an analgesic selected from codeine, prodrugs, morphine, hydromorphone, propoxyphene, hydrocodone, oxycodone, meperidine, methadone, and fentanyl; and
an antibiotic selected from erythromycin, bacitracin, neomycin, penicillin, polymyxin B, tetracyclines, viomycin, chloromycetin, streptomycins, cefazolin, ampicillin, azactam, tobramycin, clindamycin and gentamycin.

34. The method of claim 30 wherein the biological additive is selected from water, saline, radio-contrast media, hyaluronic acid, a salt of hyaluronic acid, sodium hyaluronate, glucosaminoglycan, dermatan sulfate, heparin sulfate, chondroitin sulfate, keratin sulfate, synovial fluid, a component of synovial fluid, vitronectin and rooster comb hyaluronate.

35. The method of claim 22 wherein the biological treatment comprises a biologically active component selected from anti-cytokines; anti-interleukin-1 components (anti-IL-1); anti-TNF alpha; growth factors; LIM mineralization proteins; stem cell material, osteoblasts and cells containing a viral vector for osteoinductivity.

36. The method of claim 22 wherein the biological treatment comprises a biologically active component selected from bone morphogenetic proteins, platelet derived growth factor (PDGF), insulin-like growth factor (ILGF), human endothelial cell growth factor (ECGF), nerve growth factor (NGF), and vascular endothelial growth factor (VEGF).

37. The method of claim 36 wherein the biologically active component comprises at least one bone morphogenetic protein selected from BMP-2, BMP-3, BMP-4, BMP-6, BMP-7, and BMP-9.

38. The method of claim 22 wherein the biological treatment comprises stem cell material selected from dedifferentiated stem cells, undifferentiated stem cells, mesenchymal stem cells, marrow-extracted stem cell material and adipose-derived stem cell material.

39. The method of claim 22 wherein the biological treatment comprises a biologically active component selected from biologic tissues, activated tissue grafts, engineered cells comprising a nucleic acid for encoding a protein or variant thereof, and a recombinant human bone morphogenetic protein.

40. A method of fusing a motion segment of a spinal column comprising:
applying a biological treatment to an intact motion segment of the spinal column to promote fusion within the intact motion segment.

41. The method of claim 40 wherein the intact motion segment joint comprises at least one of an intact facet joint and intact adjacent vertebrae.

42. The method of claim 41 further comprising injecting the biological treatment into the intact facet joint.

43. The method of claim 41 wherein the intact adjacent vertebrae comprise at least one of intact adjacent vertebral bodies and intact adjacent endplates, and further comprising injecting the biological treatment into at least one of the intact adjacent vertebral bodies or the intact adjacent endplates.

44. The method of claim 40 further comprising applying a mechanical device to the spinal column.

45. The method of claim 44 wherein the mechanical device is applied to the spinal column percutaneously.

46. The method of claim 40 wherein the mechanical device is applied to at least one of an anterior region, a posterior region and a spinous process region of the spinal column.

47. The method of claim 40 wherein the biological treatment is injected into the intact motion segment through a vertebral annulus between adjacent vertebrae of the intact motion segment.

48. The method of claim 40 wherein the biological treatment is injected into the intact motion segment through a bony portion of the intact motion segment.

49. The method of claim 40 further comprising:
causing damage to at least a portion of the intact motion segment sufficient to induce a healing response in the intact motion segment.
